(19) United States
(12) Patent Application Publication
(10) Pub. No.: US 2006/0241392 A1
(43) Pub. Date: Oct. 26, 2006

(54) Method and apparatus for wireless monitoring of subjects within a magnetic field

(76) Inventors: Igor Feinstein, Jersey City, NJ (US); James Kierstead, Patchogue, NY (US); Helene Benveniste, Northport, NY (US)

Correspondence Address: BROOKHAVEN SCIENCE ASSOCIATES/;BROOKHAVEN NATIONAL LABORATORY, BLDG. 475D - P.O. BOX 5000, UPTON, NY, 11973, US

(21) Appl. No.: 11/097,918

(22) Filed: Apr. 4, 2005

(51) Int. Cl. A61B 5/05

(52) U.S. Cl. 600/422

(57) ABSTRACT

An apparatus for monitoring a conscious subject, such as a rat or human, in a strong magnetic field, such as that generated by a magnetic resonance imaging (MRI) scanner, includes sensors and wireless transmitter. The sensors detect physiological parameters of the subject, and the transmitter sends a wireless signal representing the parameters. A system for monitoring a conscious subject in a magnetic field includes a wireless monitor, wireless interface, and computer. The wireless monitor includes the sensors, filters, microcontroller, and wireless transmitter. The wireless interface receives the signal from the wireless monitor and transmits a corresponding signal to the computer. A method of monitoring a conscious subject in a strong magnetic field includes disposing the sensor in the magnetic field, sensing a physiological parameter from the subject, providing a sensed signal representing the parameter, disposing a wireless transmitter on the subject, and transmitting a signal representing the sensed parameter.

This invention was made with Government support under contract number DE-AC02-98CH10886, awarded by the U.S. Department of Energy. The Government has certain rights in the invention.

### BACKGROUND OF THE INVENTION

1. Field of the Invention

The present invention relates to the monitoring of physiological parameters, such as heartbeat, and more particularly relates to wireless monitoring of physiological parameters in a magnetic field, such as that generated during a magnetic resonance imaging procedure.

The present invention relates to the monitoring of physiological parameters, such as heartbeat, and more particularly relates to wireless monitoring of physiological parameters in a magnetic field, such as that generated during a magnetic resonance imaging procedure.

2. Description of the Prior Art

The use of magnetic resonance imaging (MRI) is a popular method in the U.S. and other parts of the world for non-invasively investigating and diagnosing various diseases. Statistical data published by in-vivo research shows that over 18 million scans are performed per year in the U.S. alone. The problems of monitoring subjects undergoing MRI scanning can better be understood by summarizing the primary steps required to generate an MRI image.

1. A strong magnetic field, of at least 0.5 to 2.0 Tesla (where 1 Tesla is equal to 10,000 Gauss, the earth's magnetic field being equal to 1 Gauss) is required to align randomly oriented cell nuclei of the subject.

2. Radio frequency (RF) pulses directed at the subject are used in the presence of the external magnetic field to cause the cell nuclei to absorb more energy producing magnetic resonance. This is generally referred to as “super charging” the nuclei, which further changes the alignment from the original state.

3. The RF supercharged cell nuclei recover their original state of alignment within the magnetic field by re-emitting the absorbed RF energy. The RF signal emitted by each tissue is proportional to the difference between the energized magnetic resonance states and the original alignment states. Tissue imaging contrast develops as a result of the different rates of realignment.

4. Time varied magnetic field (TVMF) gradients are briefly applied to spatially encode the RF signals emitted from the subject's tissues.

5. RF coils in the MRI pick up the spatially encoded RF signals emitted from the tissues and are transformed by a computer into two- or three-dimensional images. The strong magnetic field, RF pulses, and/or TVMF gradients are generally referred to as “the MRI environment”.

Three broad categories of problems are typically experienced when monitoring the vital signs of subjects in an MRI: 1) MRI environment induced interference in the vital sign monitoring equipment; 2) inadequate monitoring of respiration or other physiological parameters; and 3) use of conventional electrocardiogram (EKG or ECG) electrodes and leads that cause burns to the subject and shadows in the images. Each of these problems has been addressed in light of the monitoring equipment.

As a brief introduction, an EKG measures changes in electrical potential caused by currents generated by the myocardium. This electrical activity is typically represented by PQRST waveforms. The P wave reflects atrial depolarization, the QRS complex represents ventricular depolarization, and the T wave represents ventricular repolarization. Repolarization is a process that occurs in many cells when the electrical potential across the cell membrane returns from the value during the action potential to that of the resting state, that is, the resting membrane potential. Although the EKG shows heart rate and rhythm, and can indicate myocardial damage, it does not directly provide information on the adequacy of contraction.

At the end of each EKG lead is an electrode that measures the small potential difference produced as a result of the heart's electrical activity. By measuring, for example, the rate, rhythm, impulse axis, hypertrophy, and infarction, information about the condition of the heart can be determined.

Medical research communities around the world have begun to perform MRI studies on conscious, rather than anesthetized animals, such as rats. It has become clear that traditional methods of monitoring the vital signs of such animals are impractical, particularly in high-field MRI, where many limitations on monitoring equipment exist. Even though the animal would be awake, and would thus not be in danger of death, disregarding vital signs is not an option since many MRI experiments, especially when performed on conscious animals, are correlated to vital sign data in order to interpret the resulting data. Since the animal is conscious during the experiment, it cannot be connected to wired, vital sign monitoring systems that are currently available. Such wiring would stress the animal and cause unacceptable deviations in experimental results.

Therefore, there is a significant need in the prior art for a non-invasive, self-contained system, which would be able to monitor the vital signs of a conscious small animal.

A solution has not yet been found to this specific problem. Two conventional systems include a wired MRI-compatible system, which is available from SA Instruments, Inc., 65 Main Street, Stony Brook, N.Y. 11790, and a wireless system that only monitors bioelectrical activity (RatPaak™), which is available from Cleveland Medical Devices, 4415 Euclid Ave, Cleveland, Ohio 44103. The wired system has the obvious disadvantage of being wired and, despite its description as being reliable in an MRI environment, cannot be used on conscious animals due to the size and weight of the monitoring equipment, as well as the invasive nature of several of its sensors. The wireless system only monitors a limited set of bioelectric signals, which cannot provide an acceptably complete vital sign profile. In addition, the wireless system is not designed to work in extreme environments, such as an MRI scanner.

Therefore, there is a distinct need in the prior art for an extremely compact, lightweight, wireless system that is adapted to reliably monitor a complete range of vital signs of a conscious small animal in a strong magnetic field, such as that generated in an MRI environment.

### SUMMARY OF THE INVENTION

The present invention, which addresses the needs of the prior art, relates to an apparatus for monitoring a conscious subject, such as a rat or human, in a strong magnetic field, such as that generated by a magnetic resonance imaging (MRI) scanner. The apparatus includes a sensor and a wireless transmitter. The sensor is adapted to detect a physiological parameter associated with the subject while being disposed in the magnetic field. The sensor outputs a sensed signal representative of the physiological parameter and is substantially made from at least one of non-ferromagnetic, non-metallic, non-ferrous, non-ferritic, and non-magnetic materials.

The wireless transmitter is responsive to the sensed signal, and is adapted to wirelessly transmit a signal representative of the sensed signal. The wireless transmitter is disposed on the subject in the magnetic field and is substantially made from at least one of non-ferromagnetic, non-metallic, non-ferrous, non-ferritic, and non-magnetic materials. The sensor may be adapted to detect an electrocardiogram (EKG) signal, electroencephalogram (EEG) signal, electromyogram (EMG) signal, electrooculogram (EOG) signal, pulse oximetry, respiration, blood pressure, and/or temperature.

The present invention also relates to a system for monitoring a conscious subject in a strong magnetic field, which includes a wireless monitor, wireless interface, and computer. The wireless monitor includes the sensor and wireless transmitter. The wireless interface is adapted to receive a first transmitted signal from the wireless monitor and transmit a second transmitted signal representative of the first transmitted signal. The computer is adapted to receive the second transmitted signal.

The computer may be adapted to process, tabulate, graph, display, analyze, detect errors, correct errors, filter, and/or format information associated with the second transmitted signal. The wireless interface may be adapted to transmit a third transmitted signal and the wireless monitor may include a wireless receiver adapted to wirelessly receive the third transmitted signal from the wireless interface. The wireless receiver may be disposed on the subject in the magnetic field, and the wireless receiver may be made substantially from at least one of non-ferromagnetic, non-metallic, non-ferrous, non-ferritic, and non-magnetic materials.

The present invention further relates to a method of monitoring a conscious subject in a strong magnetic field, which includes disposing the sensor in the magnetic field, sensing a physiological parameter associated with the subject by the sensor, providing a sensed signal representative of the physiological parameter from the sensor, disposing a wireless transmitter responsive to the sensed signal on the subject in the magnetic field, and transmitting a first transmitted signal representative of the sensed signal from the wireless transmitter.

Processing the sensed signal may include digitizing the sensed signal, packetization of information associated with the sensed signal, and/or formatting information associated with the sensed signal. The method may also include receiving the first transmitted signal by a wireless interface, transmitting a second transmitted signal representative of the first transmitted signal from the wireless interface, and/or receiving the second transmitted signal by the computer. The computer may be adapted to perform processing, tabulating, graphing, displaying, analyzing, detecting errors, correcting errors, filtering, and/or formatting information associated with the second transmitted signal. The method may also include transmitting a third transmitted signal by the wireless interface and receiving the third transmitted signal by a wireless receiver in the wireless monitor.

These and other objects, features, and advantages of this invention will become apparent from the following detailed description of illustrative embodiments thereof, which is to be read in connection with the accompanying drawings.

### BRIEF DESCRIPTION OF THE DRAWINGS

FIG. 1 is a block diagram of a system formed in accordance with the present invention for wirelessly monitoring physiological parameters of a subject, such as a rat or human, within a strong magnetic field, such as that generated in a magnetic resonance imaging (MRI) environment.

FIG. 2 is a block diagram of a first embodiment of a wireless monitor in accordance with the present invention for use in the system shown in FIG. 1.

FIG. 3 is a block diagram of a second embodiment of the wireless monitor in accordance with the present invention for use in the system shown in FIG. 1.

FIG. 4 is a schematic diagram of the second embodiment of the wireless monitor shown in FIG. 3.

FIG. 5 is a schematic diagram of the first embodiment of the wireless monitor shown in FIG. 2.

FIG. 6 is a block diagram of a wireless interface in accordance with the present invention for use in the system shown in FIG. 1.

FIGS. 7a and 7b is a relational flowchart of a method in accordance with the present invention for wirelessly monitoring physiological parameters of a subject in a strong magnetic field.

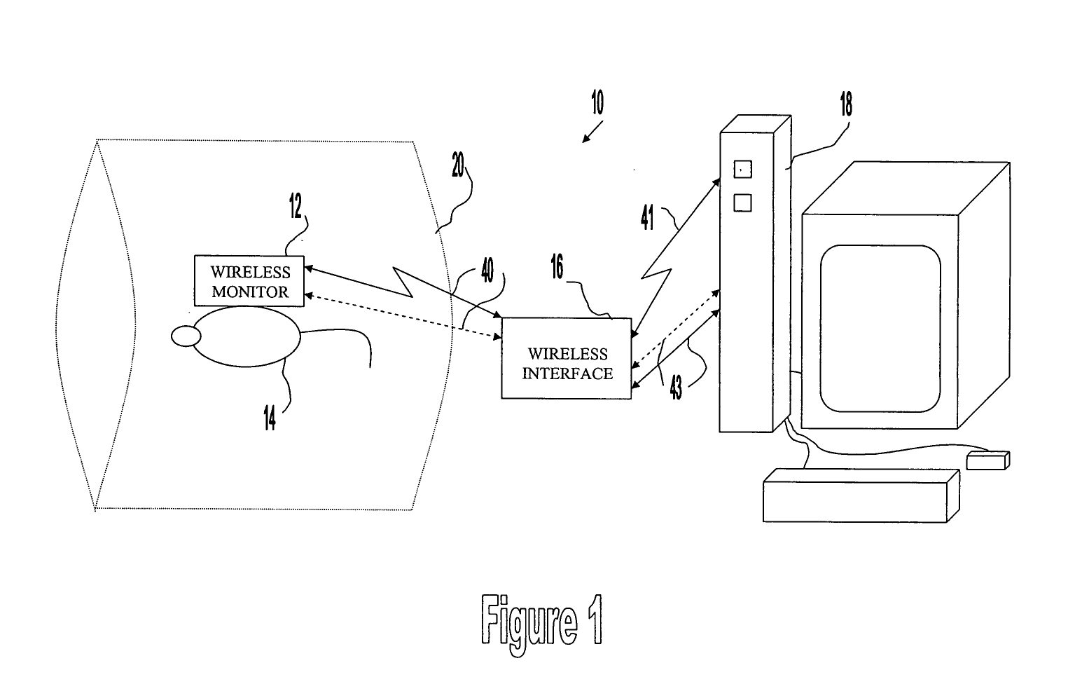

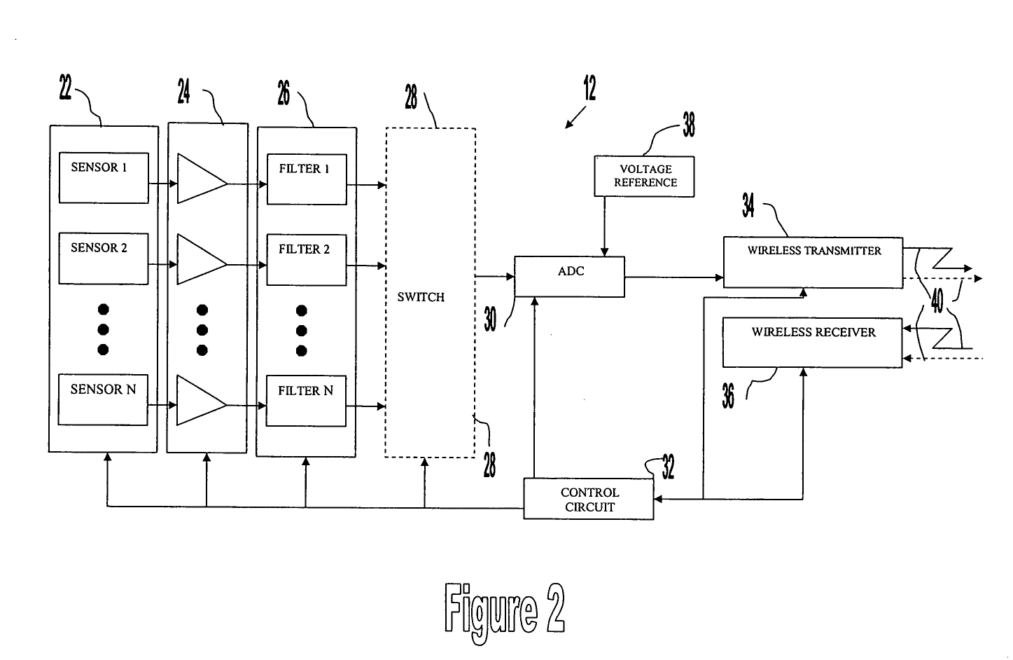

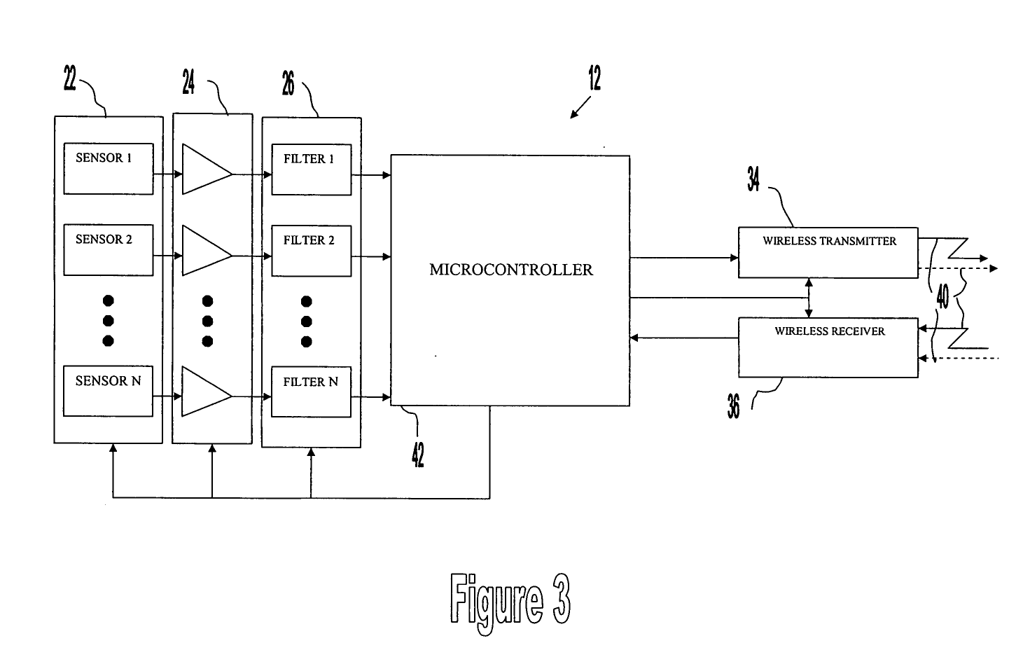

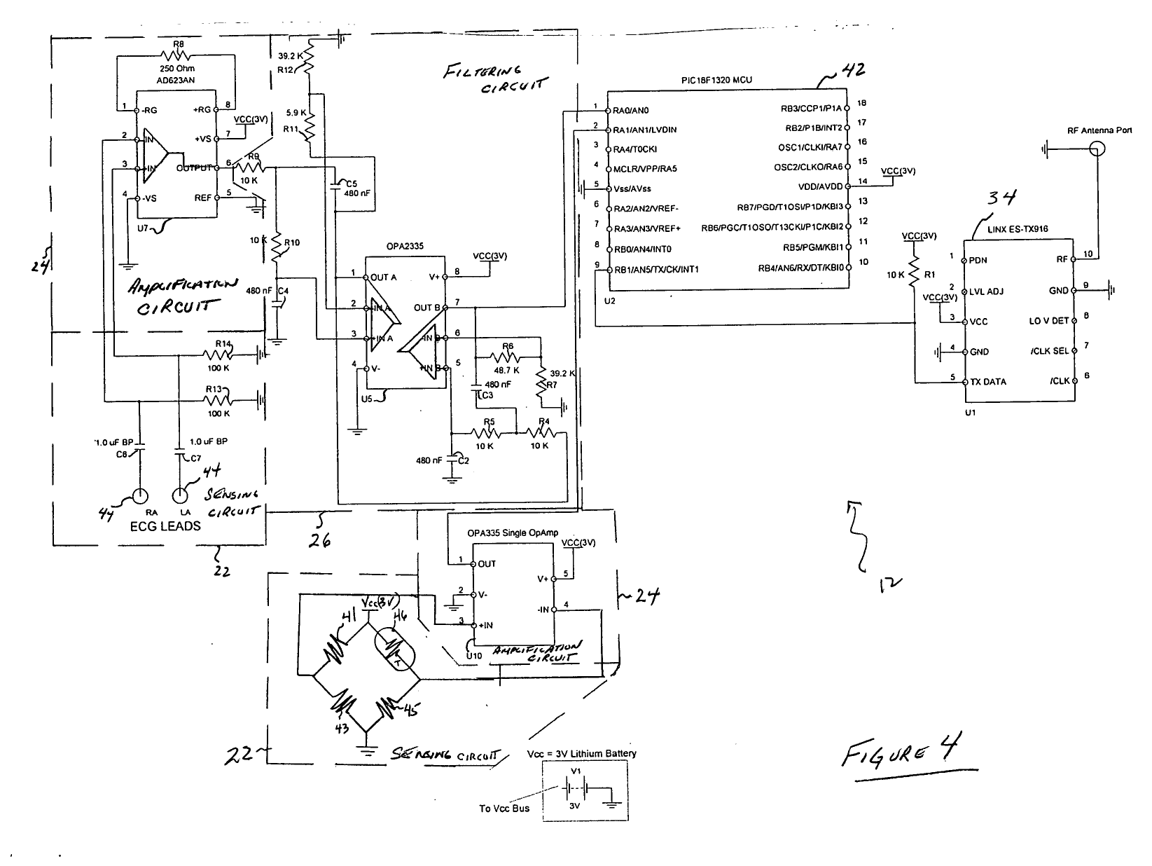

An assembly code listing of a program to direct the operation of a microcontroller in the wireless monitor shown in FIG. 4 is incorporated in this document as an appendix.

### DETAILED DESCRIPTION OF THE PREFERRED EMBODIMENTS

A wireless monitoring system 10 formed in accordance with the present invention is shown in FIG. 1 and preferably operates by obtaining signals from sensors, such as electrocardiogram (EKG or ECG) electrodes and/or a thermistor. Ambient radio frequency noise is preferably eliminated by filters in a wireless monitor 10. The wireless monitor 10 is preferably strapped, secured, and/or mounted on a subject, such as a conscious human or rat 14, which permits the subject to move about substantially freely, normally, naturally, and unencumbered.

The filtered signal is preferably converted from analog to digital form in the wireless monitor 10 and transmitted by a wireless transmitter therein to a receiver in a wireless interface 16, which is preferably about fifty (50) feet away from the monitor 12. The wireless interface 16 preferably converts information in the signal received from the wireless monitor 12 to a form that is compatible with a personal computer 18, and retransmits the information to the computer 18 through wired 41 or wireless 43 signals. Once the information is received by the computer 18, virtually any visualization or analysis can be performed on the data.

The wireless monitor 12 of the present invention is preferably optimized for use within an extremely strong magnetic field of 0.5 Tesla or more, such as that generated in a magnetic resonance imaging (MRI) scanner 20, by taking several key precautions. First, substantially non-ferromagnetic, non-metallic, non-ferrous, non-ferritic, and/or non-magnetic materials are preferably used in the wireless monitor 12, such as aluminum and copper. A-ferromagnetic material is defined herein as material that exhibits polarization by application of a magnetic field that is retained unless disturbed. R. Graf, Modern Dictionary of Electronics, 6^{th} edition, p. 370 (1984). Ferrites interfere with circuit operation and pose a significant hazard caused by their attraction to the magnetic field. A ferrite is defined herein as a magnetic material having high resistivity, including ferric oxide combined with one or more metals. R. Graf, Modern Dictionary of Electronics, 6^{th} edition, p. 368 (1984). However, using strictly non-magnetic materials is difficult since most materials have at least some trace of ferromagnetic components. A preferred method of testing whether a particular item is suitable for incorporation into the wireless monitor is to affix that item to a block of inert material, such as wood, that approximates the size and weight of the subject and note whether the forces exerted on the item by the magnetic field are acceptable.

Second, filters in the wireless monitor 12 preferably exhibit a very narrow bandpass range in order to transmit only those frequencies of interest while excluding ambient background noise which, if not filtered, could easily mask the desired signal. Thus, the filters preferably include a sharp cutoff frequency in order to eliminate undesirable interference.

The wireless monitor 12 preferably inputs signals from sensors, such as EKG electrodes to determine cardiac function, and a thermistor to determine body temperature. The rates of signal acquisition are preferably governed by a clock frequency that varies the specific acquisition rate based on the sensor being sampled. A sensor signal (i.e. EKG) is preferably passed through a fourth-order low-pass filter and a first-order high-pass filter to remove direct current (DC) bias and high-frequency ambient noise. The filtered signal is then preferably digitized to improve transmission stability and reduce errors during transmission.

The analog-to-digital conversion rate is preferably synchronized by a clock signal provided by an integrated circuit within the wireless monitor 12. Following digitization, the signal is wirelessly transmitted by, for example a radio frequency signal and/or infrared signal 40, to the receiver in the wireless interface 16, which provides the information to the computer 18 by wired means 41, such as RS-232 signals, universal serial bus (USB) signals, and/or the like, and/or wireless means 43, such as radio frequency signals, infrared signals, and/or the like. Acquisition, conversion, and transmission of information obtained by the sensors in the wireless monitor 12 are preferably coordinated by a microcontroller therein, which also preferably packetizes the information so that the wireless interface 16 can more accurately receive and differentiate information between various sensors being sampled from the subject 14.

The wireless interface 16 preferably converts the received data to a format suitable for transmission over a USB interface, and transmits the information to the computer 18, which may perform further error correction, filtering, visualization, and analysis. A real-time graph corresponding to each sensor channel is preferably generated by the computer 18 for monitoring and analysis purposes.

Use of substantially non-ferromagnetic, non-metallic, non-ferrous, non-ferritic, and/or non-magnetic components in the wireless monitor 12 essentially eliminates the use of inductive elements. In addition, even non-magnetic silicon integrated circuits are often housed in metallic packages that contain ferrites. Many integrated circuits are unavailable in completely non-metallic packages, which further limits the range of possibilities. Since subcutaneous metallic electrodes typically used on small animals for EKG monitoring cannot be used in an MRI environment, a different type of sensor that is safe to the animal within the magnetic field must be used.

The filters within the wireless monitor 12 preferably include a bandpass filter having a range of about 1.4 Hz to 30.0 Hz. The filter preferably includes a first-order high-pass filter to remove low frequency/DC components and a fourth-order low-pass filter to remove high-frequency components. Since the pulse of a rat is of the order of about 4 HZ, the low-pass filter preferably exhibits a sharp cutoff at about 30.0 Hz. The selected bandpass region preferably includes a frequency range in which operation is stable and substantially noiseless.

Transmissions between the wireless monitor 12 and wireless interface 16 are preferably provided at frequencies that will not interfere with the MRI scanner and that will not be interfered with by the MRI scanner. Since the resonant frequency of the MRI scanner is about 400 MHz, commercially available transmitters operating in the range of 400 MHZ±200 MHZ cannot be used due to interference. Accordingly, a transmitter operating at about 916.5 MHz, which is between the second and third harmonic frequencies of the MRI scanner, is preferably incorporated in the wireless monitor 12. Noise remaining at the preferred transmission frequency of 916.5 MHz is preferably reduced or eliminated through the use of filters, the proper choice of antennas, and shielding.

FIG. 2 shows a block diagram of a first embodiment of the wireless monitor 12 shown in FIG. 1. The wireless monitor 12 preferably includes one or more sensors 22 that detect various physiological parameters from the subject 14 (such as cardiac function (EKG), brain wave activity (EEG), muscle activity (EMG), eye activity (EOG), temperature, pulse oximetry, blood pressure, and respiration) and output corresponding signals to amplifiers 24.

The amplified signals are preferably applied to a filtering stage 26, which may include an independent filter for each of the amplified signals, and the outputs of the filtering stage 26 are preferably applied to a switch or multiplexer 28. The multiplexer 28 provides the capability to select one of the filtered outputs for input to an analog-to-digital (ADC) converter 30 in response to a control signal provided by a control circuit 32. The control circuit 32 may provide control signals to the sensors 22, amplifiers 24, filters 26, ADC 30, a wireless transmitter 34, and a wireless receiver 36.

The output selected by the multiplexer 28 is preferably applied to the ADC 30, which digitizes the analog signal in accordance with a voltage reference 38 and outputs the digitized signal to the wireless transmitter 34. The wireless transmitter 34 then preferably outputs the wireless signal 40 to the wireless interface 16 shown in FIG. 1. The wireless receiver 36 may receive wireless signals 40 from a source, such as the wireless interface 16 and provide information from these signals to the control circuit 32 for use in the wireless monitor 12.

FIG. 3 shows a second and preferred embodiment of the wireless monitor 12 shown in FIG. 1. The second embodiment of the wireless monitor is similar to the first embodiment shown in FIG. 2, except that a microcontroller 42 has been incorporated in the design and substantially replaces the multiplexer 28, control circuit 32, ADC 30, and voltage reference 38. This modification reduces the number of active components in the wireless monitor 12, reduces the overall size of the wireless monitor, reduces the power requirement of the wireless monitor, and permits simplified expansion of the monitoring capability and straightforward modification of the parameters monitored. The microcontroller 42 may be implemented by a microprocessor, application specific integrated circuit (ASIC), field programmable gate array (FPGA), and/or programmable device while remaining within the scope of the present invention.

FIG. 4 is a schematic diagram of a preferred implementation of the wireless monitor 12 shown in FIG. 3. The function of the wireless monitor 12 is substantially linear, and thus may be broken down into several discrete steps. These steps include data acquisition, amplification, filtering, analog-to-digital conversion, packetization, and transmission.

Data acquisition in the sensing circuit 22 is preferably performed by two electrodes 44 for the EKG signal, and by a thermistor 46 for body temperature. The electrodes 22 are each preferably connected in series with 1.01 μF capacitors C8, C7. The remaining terminals of the capacitors C8, C7 are each preferably connected to 100 kΩ resistors R13, R14, which are each then connected to ground. The thermistor 46 is preferably connected in a resistive bridge circuit, which includes resistors 41, 43, 45 and outputs voltage as a function of the resistance across the thermistor 46 to an amplification circuit 24 for sensing body temperature. The acquired EKG and temperature signals are preferably applied to separate amplification circuits 24 to allow for variations in the amplifier specifications applied to each type of signal.

The amplification circuit 24 for the EKG signals preferably includes an instrumentation amplifier U7, which is available as part number AD623AN from Analog Devices, One Technology Way, P.O. Box 9106, Norwood, Mass. 02062. The EKG signals from the sensing circuit 22 are preferably applied to pins 2 and 3 of the amplifier U7. The output of the amplifier U7 is provided at pin 6, which is preferably connected in series with a 10 kΩ resistor R9. The remaining terminal of resistor R9 is connected to the series combination of a 10 kΩ resistor RIO and a 480 nF capacitor C4, which is connected to ground. Pins 1 and 8 of the amplifier U7 are preferably connected through a 250Ω resistor R8 to provide a gain of about 400 from the amplifier U7.

The bridge circuit, which includes resistors 41, 43, 45, and thermistor 46, is preferably connected to the amplification circuit 24 for sensing body temperature at pins 3 and 4 of operational amplifier U10. Operational amplifier U 10 is available as part number OPA2335 from Texas Instruments, P.O. Box 655303, Dallas, Tex. 75265.

The filtering circuit for the EKG signals preferably includes a first-order high-pass filter that substantially eliminates DC bias, which is a common occurrence in EKG monitoring applications. The cutoff frequency for the high-pass filter is preferably about 1.6 Hz, which is low enough to ensure that the EKG signal (which is about 4 Hz for a rat) is not affected. Since the MRI environment, in which this device may operate, is extremely noisy, the design of a selective and robust low-pass filter is important.

Thus, the filtering circuit 26 preferably incorporates a fourth-order low-pass Butterworth filter with a cutoff frequency of about 30.0 Hz. This ensures that substantially all high-frequency emissions from the magnet, fluorescent lights, and common 60 Hz appliances are substantially eliminated. The high-pass filter is preferably implemented using pins 1-3 of an operational amplifier U5, which is available as part number OPA2335 from Texas Instruments, P.O. Box 655303, Dallas, Tex. 75265, as well as a 39.2 kΩ resistor R12, 5.9 kΩ resistor R11, 480 nF capacitor C5, 10 kΩ resistor R9, 10 kΩ resistor R10, and 480 nF capacitor C4, as shown in FIG. 4. Similarly, the low-pass filter is preferably implemented using pins 5-7 of the operational amplifier U5, a 39.2 kΩ resistor R7, 48.7 kΩ resistor R6, 480 nF capacitor C3, 10 kΩ resistor R4, 10 kΩ resistor R5, and 480 nF capacitor C2, as is also shown in FIG. 4.

The output of the filtering circuit 26 for the EKG signals and the output of the amplification circuit 26 for the temperature signal are preferably applied to pins 1 and 2, respectively, of a microcontroller 42, which is available as part number PIC18F1320 from Microchip Technology, Inc., 2355 West Chandler Blvd., Chandler, Ariz. 85224, the datasheet for which is incorporated herein by reference. The microcontroller 42 preferably performs analog-to-digital conversion and digital serialization of the sensor information.

The microcontroller 42 preferably includes a 10-bit ADC with a step size of about a millivolt, which provides sufficient resolution for applications involving the vital signs of small animals. Software preferably controls synchronization of the ADC, channel switching (such as between the EKG, temperature, and any additional sensor signals) packetization, and transmission as further described herein.

After the data has been digitized by the microcontroller 42, it is preferably packetized. This enables errors in transmission to be readily detected by the receiver in the wireless interface and, if an error is found in any particular packet, only the corrupted packet is treated as invalid rather than an entire data stream. Packetization also enables more efficient utilization of the available bandwidth while simplifying the design of the receiver.

The packetized data is preferably provided at pin 9 of the microcontroller 42, which is connected to pin 5 of the wireless transmitter 34. The wireless transmitter is available as part number TXM-916-ES from Linx Technologies, Inc. 575 S.E. Ashley Place, Grants Pass, Oreg. 97526. Pin 5 of the wireless transmitter 34 is also preferably connected to the voltage source Vcc/3v through a 10 kΩ resistor R1. The output of the wireless transmitter 34 is provided at pin 10, which is connected to an antenna. The wireless receiver 36 shown in FIG. 3 has not been shown in FIG. 4, but may be implemented using, for example, part number RXM-916-ES available from Linx Technologies, by means well known in the art to facilitate reception, as well as transmission of information by the wireless monitor 12.

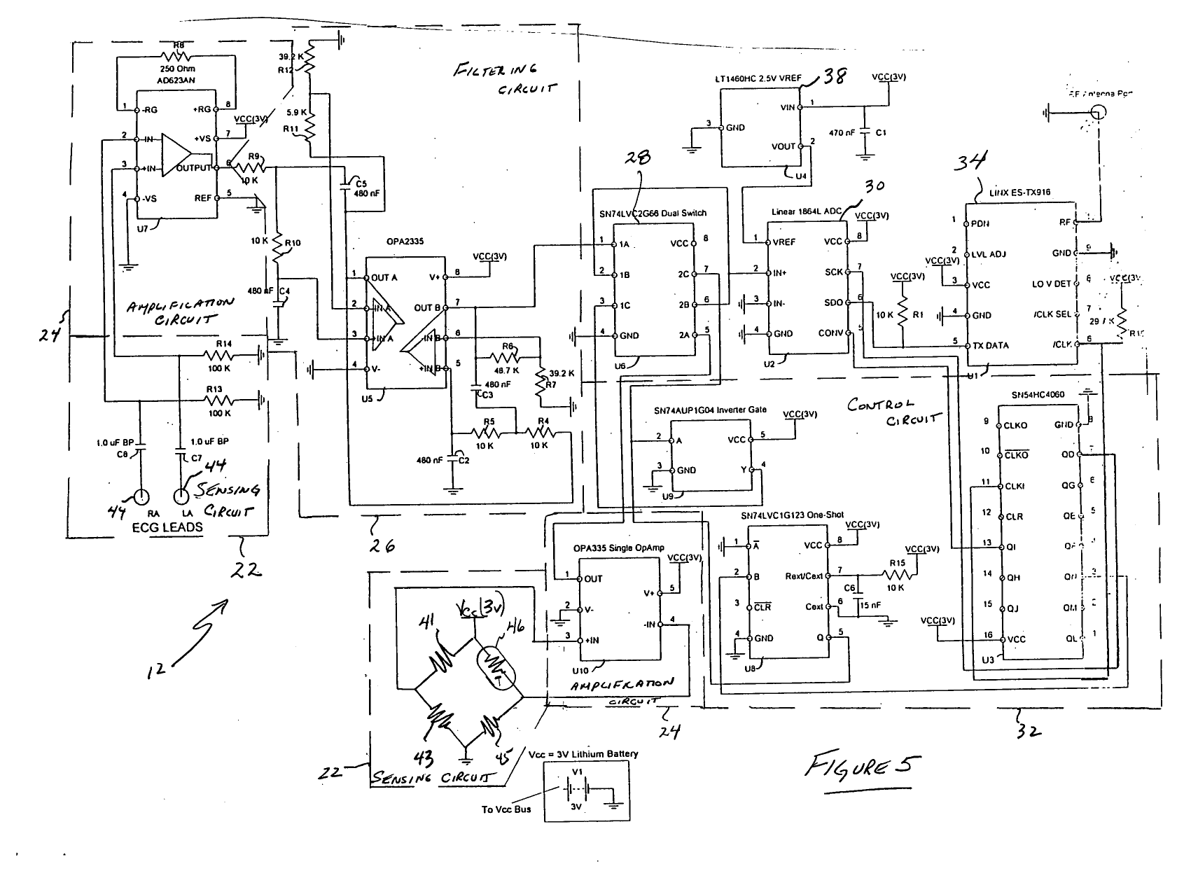

FIG. 5 is a schematic diagram of the first embodiment of the wireless monitor 12 shown in FIG. 2. This embodiment is similar to the second embodiment, except that the microcontroller 42 has been replaced by a multiplexer 28 or bilateral switch, which is available as part number 74LVC2G66 from Philips Semiconductor, Inc., Los Angeles, Calif. 100068, inverter gate U9, which is available as part number 74AUP1G04 from Texas Instruments, P.O. Box 655303, Dallas, Tex. 75265, monostable multivibrator or one-shot U8, which is available as part number 74LVC1G123 from Texas Instruments, 14-stage asynchronous counter U3, which is available as part number 54HC4060 from Texas Instruments, ADC 30, which is available as part number TC1864L from Linear Technology Corporation, 1630 McCarthy Blvd. Milpitas, Calif. 95035, and 2.5 v voltage reference U4, which is available as part number LT1460HC from Linear Technology Corporation.

The switch U6 essentially enables the selection of one of the sensor signals to be applied to the ADC U2 at any given time in accordance with timing and control signals generated by the one-shot U8, counter U3, and inverter U9 by means known in the art.

The counter 32 is a frequency counter that preferably inputs a primary clock frequency from the wireless transmitter 34 and divides it into three (3) additional frequencies. These frequencies preferably include a baud rate, an overall sensor sampling conversion rate, and a specific sampling rate for the thermistor. The ADC 30 is the analog to digital converter. The ADC 30 preferably receives an analog input on pin 2, the baud rate on pin 7 and the conversion rate on pin 5. The voltage reference 38 preferably provides a stable voltage reference to the ADC 30 for conversion.

The switch 28 is an analog input select for the input to pin 2 of the counter 32. The switch 28 is preferably controlled by the one-shot U8 and inverter U9. The one-shot U8 preferably provides a pulse having a width slightly smaller than the width of the sampling rate, and is triggered by a negative edge from the counter 32, which defines the sampling rate for body temperature. The inverter U9 is preferably used to ensure that either the temperature or the EKG, but not both, are present on the input channel of the ADC 30. This embodiment is shown with two (2) sensor inputs, but may be expanded to accommodate additional sensors by means well known in the art.

The embodiment of the wireless monitor 12 shown in FIG. 4 represents a substantial improvement over that shown in FIG. 5 in that the microcontroller 42 is capable of performing the tasks of six separate components shown in FIG. 5. This significantly reduces power consumption and the size of the monitor 12, while facilitating expansion capabilities to handle a greater number of sensor channels.

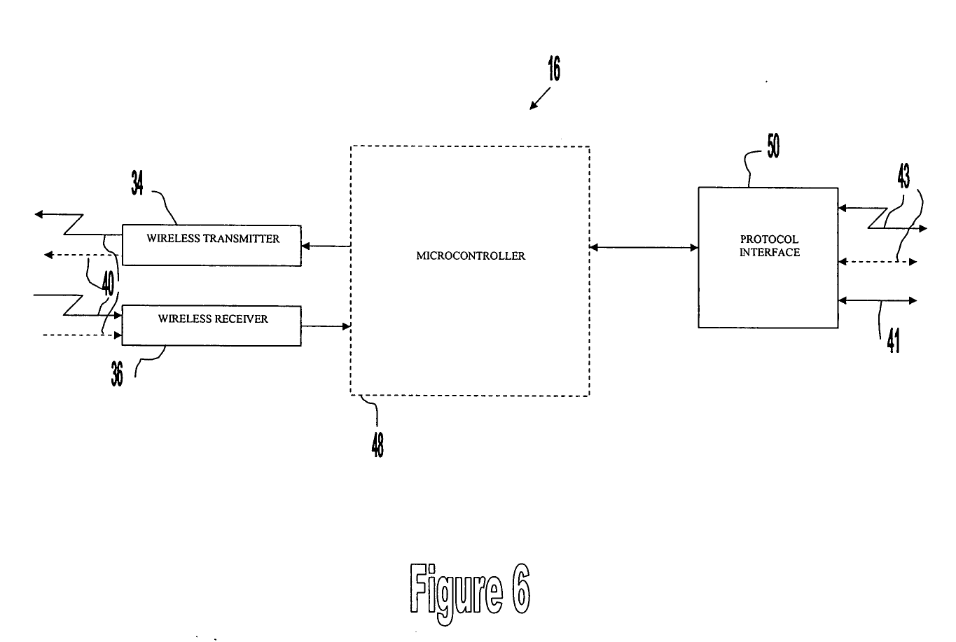

FIG. 6 is a block diagram of the wireless interface 16 shown in FIG. 1. The wireless interface 16 preferably includes a wireless receiver 36 and a wireless transmitter 34, which may be implemented using the same or similar components as those shown in FIGS. 2-5, a microcontroller 48, and a protocol interface 50. The wireless receiver 36 is preferably implemented using a receiver module, which is available as part number RXM-916-ES from Linx Technologies. The signals 40 transmitted by the wireless monitor are received and demodulated by the wireless receiver 36 and provided to the microcontroller 48, which may perform additional formatting in preparation for transfer to the computer shown in FIG. 1 by the interface circuit 50 via wired 41 and/or wireless 43 signals.

Since the wireless data link between the wireless monitor and wireless interface is preferably asynchronous, the microcontroller 16 preferably performs baud rate detection and synchronizes the clock in the wireless receiver 36 to the received data stream. Similarly, the microcontroller 12 in the wireless monitor of FIG. 3 preferably performs baud rate detection and synchronizes the clock in the wireless receiver 36 of the monitor to the data stream received from the wireless interface. This is described in more detail below. Thus, the microcontroller 16 preferably synchronizes the data stream provided to the computer 50 to the baud rate of the data stream received from the wireless monitor to ensure an error-free data transfer.

As stated above, two problems arise in the transmission of digital data in a noisy environment, such as that between the wireless monitor 12 and the wireless interface 16. One is determining the baud rate of the transmitted data by the receiver. The other is the loss of data through interference.

The baud rate is preferably detected by the wireless receiving interface using a repetitious data transmission structure and an auto-baud detect technique. This technique allows for drift in the baud rate clock in the transmitter due to, for example, changes in temperature and voltage, and eliminates the need for additional components required to stabilize the clock.

It is also anticipated that data will occasionally be corrupted by noise or interference. To limit data loss, data are preferably transmitted in packets with a well-defined start or header sequence. As an example, each sensor measurement is preferably encoded into 10 bits, which require two 8-bit serial bytes. Each unit of sensor data then preferably utilizes only the rightmost ten (10) bits of the sixteen (16) total bits. The remaining unused six (6) bits are preferably set to zero.

Thus, a header defined, for example, as a byte of eight (8) “zero” or “low” bits (OOH) followed by a byte of eight (8) “one” or “high” bits (FFH) is unique and should not be duplicated during the transfer of sensor data within the packet. The two (2) header bytes are preferably followed by a synchronization byte of hexadecimal fifty-five (55H) or Binary 01010101, which is preferably used to synchronize the clock in the receiver. The sensor data is preferably transmitted after the synchronization byte.

Sensor data preferably includes body temperature, EKG data, as well as other measurements. As indicated above, the microcontroller 42 shown in FIG. 3 is preferably implemented using the PIC18F1320 shown in FIG. 4, which incorporates four (4) analog-to-digital conversion channels, and thus can accommodate up to four (4) different sensor signals with a resolution often (10) bits. The resolution, as well as the number of analog-to digital conversion channels may be increased or reduced by selecting a different microcontroller.

Since temperature data does not change as rapidly as EKG data, the temperature data is preferably transmitted at a much slower rate. Thus, inclusion of one unit of temperature data per data packet, which occupies two (2) bytes, the temperature can be measured at a rate of more than 10 times per second, which is more than adequate. The remaining data in the data packet could then be devoted to additional sensor data, such as EKG data. Accordingly, a typical data packet may constructed as follows:

Header bytes:

Hexadecimal 00—all low;

Hexadecimal FF—all high; and

Hexadecimal 55—which is preferably used for baud rate detection.

Data bytes:

Temperature low byte;
Temperature high byte;
1st EKG low byte;
1st EKG high byte;
.;
.;
.;
255th EKG low byte; and
255th EKG high byte;

Thus, the packet described above includes a total of 515 bytes. Following measurement and transmission of a data packet, another data packet is preferably transmitted with the header bytes and no gap in data transmission. For flexibility, the size and content of the data packet can be readily changed. An example of a program in assembly code for the microcontroller 42 shown in FIG. 4 is incorporated herein as an appendix. This program preferably acquires temperature and EKG data, serializes the data, and provides the data on output pin 9 of microcontroller 42 at a selected baud rate.

As indicated above, the preferred data transmission scheme enables the clock in the wireless receiver of the wireless interface 16 shown in FIG. 1 to be synchronized from the transmitted packets, which is referred to herein as “autobaud detection”. Since the wireless transmission between, for example, the wireless monitor 12 and wireless interface 16 is preferably asynchronous, the receiver does not know the data transmission rate or baud rate prior to transmission of the signal. This presents a problem in distinguishing separate bits in the received signal, which can easily result in data being lost, even with slight variations in the baud rate.

Thus, autobaud detection is preferably implemented in the microcontroller 42 in the wireless interface 16. The serial port of the microcontroller 48, which is also preferably implemented using the PIC18F1320 from Texas Instruments, includes a feature that allows it to detect the baud rate of an incoming transmission by detecting a predefined sequence of Hexadecimal fifty-five (55H) or binary 01010101. When the microcontroller 48 in the wireless interface 16 detects this byte, it automatically detects the baud rate and synchronizes its internal clock accordingly. This ensures the synchronization of data transmissions between the wireless monitor 12 and wireless interface 16, and enables recovery of data even if the baud rate is altered by the transmitter, which is a common occurrence in response to variations in voltage or temperature. Transmissions from the wireless interface 16 to the wireless monitor 12 and between the wireless interface 16 and computer 18 may be performed in a similar manner to that described herein.

By placing the autobaud detect byte in each data packet, it is ensured that if the baud rate changes rapidly, the receiver will be robust enough to detect this change and synchronize itself with the new baud rate. This will minimize data losses and provide for greater stability of the wireless link.

The protocol interface 50 preferably inputs information from the microcontroller 48 and formats it in accordance with, for example, universal serial bus (USB) and/or RS-232 protocols, for transmission to the computer. The protocol interface preferably converts information received from the microcontroller 16 to USB format using a USB module, which is available as part number SDM-USB-QS-S from LINX Technologies that converts an asynchronous serial transmission to a signal that can be transmitted on a USB bus. The USB module is preferably connected to a standard USB jack for interfacing with the computer 50. Use of the USB interface is preferable to a traditional serial interface due to the higher flexibility and wider availability of the USB interface on commercially available personal computers. In addition, the USB interface enables identification of the source of transmitted information to any computer connected thereto.

The wireless interface 16 preferably transmits information received from the wireless monitor with a minimum of additional processing to ensure adequate throughput for a multi-channel sensing system. This simplicity of operation also ensures that there are no unnecessary complications in the transmission operation that may corrupt the information.

The wireless interface 16 may also receive information from the computer or other sources by wired 41 or wireless 43 means, and transmit wireless signals 40 from the wireless transmitter 34 to the wireless monitor shown in FIG. 1 or other destinations by wireless means 40. As with the microcontroller 48 in the wireless monitor, the microcontroller 42 in the wireless interface 16 is preferably implemented using part number PIC18F1320, which is available from Microchip Technology, but may also be implemented by a microprocessor, application specific integrated circuit (ASIC), field programmable gate array (FPGA), and/or programmable device while remaining within the scope of the present invention.

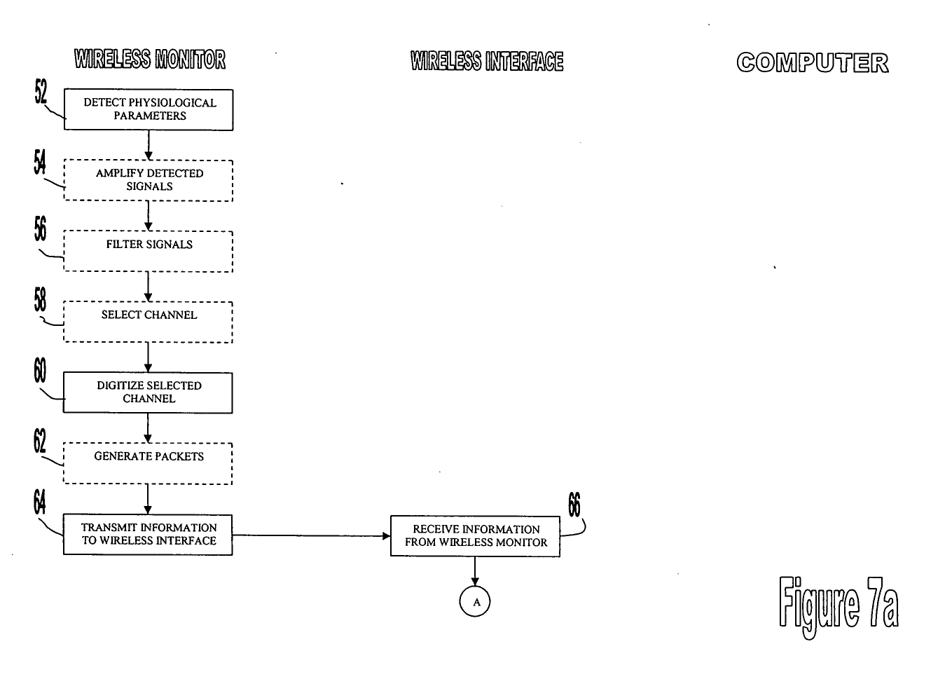

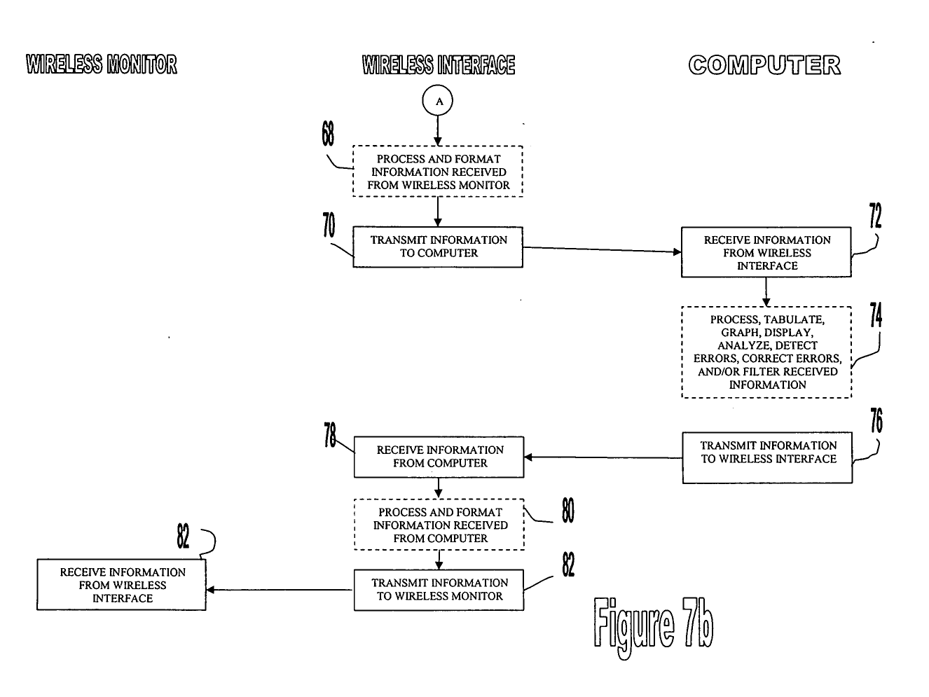

FIGS. 7a and 7b show a relational flowchart of the tasks preferably performed by the wireless monitor, wireless interface, and computer shown in FIG. 1. In the wireless monitor, physiological parameters are sensed or detected in step 52, and the detected signal is amplified in step 54. The signal is then filtered in step 56 to remove undesirable frequency components and noise, and a channel of interest is selected in step 58 for digitization. The selected signal is then digitized in step 60, and packets of information are generated with optional error correction and detection features in step 62.

The information is preferably transmitted by the wireless monitor in step 64 and received by the wireless interface in step 66. As shown in FIG. 7b, the wireless interface may then process and format the information in step 68, and transmit this information to the computer in step 68. The computer then receives the information in step 72, and may perform further processing, analysis, tabulation, graphing, display, error detection, error correction, filtering, and/or the like thereon in step 74.

The computer may also transmit information to the wireless interface in step 76, which is received in step 78 and optionally processed and/or formatted by the wireless interface in step 80. The information received by the wireless interface may then be transmitted to the wireless monitor in step 82 and received in step 84.

The embodiments described above are intended as examples without limiting the scope of the present invention in any way, which may incorporate any or all of the features of the exemplary embodiments, as well as the following:

1. Sensors included in the wireless monitor to detect physiological parameters associated with one or more of the following: Pulse Oximetry, Blood pressure, Respiration, EKG (heart function), EEG (brain wave activity), EMG (electrical muscle activity), and EOG (electrical eye function activity).

2. One or more of the blocks shown in FIGS. 2, 3, and 6, such as but not limited to the switch 28 in FIG. 2 and the microcontroller 48 in FIG. 6, may be omitted or placed in a different sequence from that shown, as indicated by dotted lines defining these blocks.

3. One or more of the steps shown in FIGS. 7a and 7b, such as but not limited to steps 54, 56, 58, 62, and 68, may be omitted or placed in a different sequence from that shown, as indicated by dotted lines defining the these steps.

Thus, the present invention is unique from currently available systems in at least two key ways. First, conventional wireless systems are not designed for operation in “extreme” environments, such as the MRI environment. Without special consideration in practically every step of the design process, reliable operation in the MRI environment cannot be accomplished. For example, the filters in the wireless monitor must be carefully chosen and designed so that the system will not fail in an environment with high levels of ambient electromagnetic noise while being able to filter out all undesired noise and reliably transmit acquired data to a receiver. In addition, the wireless monitor may incorporate shielding from electromagnetic radiation in the MRI environment, which is preferably designed on a unique basis for each component. Further, conventional wireless systems can only monitor a limited number of parameters, which does not provide a complete indication of vital signs.

Thus, the method and apparatus in accordance with the present invention provides a non-invasive, self-contained system, which is adapted to monitor the vital signs of a conscious small animal, such as a rat. In addition, the method and apparatus provide an extremely compact, lightweight, wireless system that is adapted to reliably monitor a complete range of vital signs for a conscious small animal in an MRI environment.

Although illustrative embodiments of the present invention have been described herein with reference to the accompanying drawing, it is to be understood that the invention is not limited to those precise embodiments, and that various other changes and modifications may be effected therein by one skilled in the art without departing from the scope or spirit of the invention.

| | |
|---|---|
| ; | Program name AD CONV.This program is designed to acquire ecg data |

and temperature data

| | |
|---|---|
| ; | through separate A to D channels and serialze the results in a |

packet

| | |
|---|---|
| ; | which will be wirelessly transmitted to a remote location. The |

serial packet is made

| | |
|---|---|
| ; | up of: |

;
;

| | |
|---|---|
| ; | 1)B 00000000 |
| ; | 2)B 11111111 |
| ; | 3)B 01010101 ;hex 55 |
| ; | 4)Temperature data high byte |
| ; | 5)Temperature data low byte |
| ; | 6)EKG High (first) |
| ; | 7(EKG Low (first) |
| ; | 8) . |
| ; | 9) . |
| ; | 10). |
| ; | n−1) Ekg High (last) |
| ; | n) Ekg Low (last) |

;

| | |
|---|---|
| ; | n = 259 for the prototype design |

;

| list p=18f1320 | |
|---|---|
| #Include <p18f1320.inc> | ;file contains addresses for register and |

bit names
;
;Start at the reset vector
;reset and interrupt vectors
;

| | | |
|---|---|---|
| TPOS | equ | 0X20 |

| TCOUN1 | equ | 0X21 | |
|---|---|---|---|
| ERR | equ | 0X22 | ;no error implemented yet |
| ; | | | |

| | |
|---|---|
| org 0x00000 | ;reset vector address |
| goto Start | |
| org 0x00008 | ;interrupt vector address |
| goto ISR | ;ISR is beginning of interrupt service routine |

| ; Start application beyond vector area | |
|---|---|
| ; code | 0x002A |

Start
;Initialize I/O pins
;Select internal clock

| | | |
|---|---|---|
| movlw | B‘01110000’ | ;Select 8MHz internal frequency |
| movwf | OSCCON | |

;
;SET UP REGISTERS FOR INTERRUPTS
;ENABLE HIGH PRIORITY AND PERIPHERAL INTERRUPTS

| | |
|---|---|
| MOVLW | B‘11000000’ |
| MOVWF | INTCON |

;
;SET UP RCON REGISTER
;

| | |
|---|---|
| MOVLW | B‘10000000’ |
| MOVWF | RCON |

;

| | |
|---|---|
| ; | CONFIGURE INPUT AND OUTPUT PORTS A AND B |

;

| clrf | PORTA | |
|---|---|---|
| clrf | PORTB | |
| movlw | B‘00111111’ | ;BITS 0 AND 2 WILL BE USED FOR ECG AND |

TEMPERATURE

| | | |
|---|---|---|
| movwf | TRISA | ;BITS 0-5 SELECTED AS INPUTS, 6-7 AS |

OUTPUTS

| | | |
|---|---|---|
| movlw | B‘11110111’ | ;BITS 1 AND 4 MUST BE = 1 FOR EUSART TO |

WORK

;PIN 1 IS TRANSMIT AND PIN 4 IS RECEIVE

| | | |
|---|---|---|
| movwf | TRISB | ;BITS 0-2 INPUTS, 3 OUTPUT, 4-7 INPUTS |

;Configure A/D channels

| | | |
|---|---|---|
| movlw | B‘11100000’ | ;BITS 5 AND 6 MUST BE = 1 FOR EUSART |

TO WORK

| | | |
|---|---|---|
| movwf | ADCON1 | ;Configure RA0-RA4 as analog |

| | |
|---|---|
| | ;USED FOR ANALOG INPUT 0,2 LOW VOLTAGE |
| DET. 1, VREF+ 3 | |
| | ;channel 0 is EKG, Channel 2 is |
| Temperature | |
| ; | |

| | | |
|---|---|---|
| movlw | B‘10101101’ | ;Right justified, SEL 12 TAD FOR |

ACQUISTION, 16 TOSC SELECTED

| | |
|---|---|
| | ;FOR CONVERSION TIME, right justified |
| fills 6 MSB with zero. | |

| | |
|---|---|
| movwf | ADCON2 |

;

| | | |
|---|---|---|
| movlw | B‘00001001’ | ;Channel 2 temperature first, Int |

voltage Ref, AD on

| | |
|---|---|
| movwf | ADCON0 |

;
;Configure transmitting,receiving,baud rate control, and baud rate words
;
;PRIORITY SET INTERRUPT FOR TRANSMITTER
;

| | | |
|---|---|---|
| MOVLW | B‘00010000’ | ;SET TRANSMITTER HIGH PRIORITY |

| | | |
|---|---|---|
| MOVWF | IPR1 | ;do it |

;
;TRANSMITTER STATUS AND CONTROL

| | |
|---|---|
| MOVLW | B‘00100100’ |
| MOVWF | TXSTA |

;
;RECEIVER STATUS AND CONTROL

| | | |
|---|---|---|
| MOVLW | B‘10000000’ | ;BIT 7 MUST BE = 1 FOR EUSART |
| MOVWF | RCSTA | |

;
;BAUD RATE CONTROL

| | |
|---|---|
| MOVLW | B‘00000000’ |
| MOVWF | BAUDCTL |

;
;BAUD RATE WORDS

| | | |
|---|---|---|
| MOVLW | B‘00001000’ | ;THE VALUES OF SPBRG AND SPBRGH |

;WILL SELECT BAUD 55555 which is within the
;the maximum value of 56000 for the transmitter

| | |
|---|---|
| MOVWF | SPBRG |
| MOVLW | B‘00000000’ |
| MOVwF | SPBRGH |

;
;END OF CONFIGURATION OF TRANSMITTER RECEIVER AND BAUD RATE
;

| | |
|---|---|
| LOOP | ;THIS IS MAIN PROGRAM LOOP, THE A TO D AND SERIAL PORTS |
| | ;ARE CONFIGURED AND IT IS TIME TO TAKE AND SEND DATA |

;
;GENERAL STRATEGY IS TO FIRST SEND 2 START BYTES FOLLOWED BY HEX 55 BYTE
;WHICH ALLOWS RECEIVING DEVICE TO DETECT BAUD RATE. AS SOON AS INTERRUPT
FOR
;AUTOBAUD BYTE OCCURS THEN THE A TO D CONVERSION WILL BE INITIATED FOR
THE TEMPERATURE
;CHANNEL. WE WILL LOAD THE TEMPERATURE DATA IN TRANSMIT AND UPON THE 2ND
INTERRUPT THE
;FIRST CONVERSION OF ECG DATA WILL START. THIS CONTINUES UNTIL WE
TRIGGER ENOUGH ECG
;CONVERSIONS FROM THE SECOND A TO D CHANNEL
;TO FINISH OFF THE REST OF THE DATA BLOCK. WHEN THE LAST BYTE IS SENT WE
WILL LOOP BACK
;TO THIS POINT AND TRIGGER ANOTHER SET OF DATA ACQUISITION.
;'
;Variables for loop control are 1) TPOS which tells us whether an
interrupt has occurred

| | |
|---|---|
| ; | 2) TCOUN1 which is the data point countdown |
| ; | |

| | |
|---|---|
| ; | TIME TO START |

;

| | | |
|---|---|---|
| MOVLW | .1 | ;TPOS=1 START BYTES TPOS=0 DATA |
| MOVWF | TPOS | ;THIS REGISTER INDICATES WHETHER WE HAVE HAD AN |

INTERRUPT

| | | |
|---|---|---|
| MOVLW | .20 | ;MOVE DECIMAL 255 INTO W |

| | | |
|---|---|---|
| MOVWF | TCOUN1 | ;MOVE INTO TEMPORARY MEMORY LOCATION, ACCOUNTS |

FOR TEMPERATURE AND ECG

| | |
|---|---|
| | ;TCOUN1 TRACKS NUMBER OF DATA POINTS NOT INCLUDING 3 |
| START BYTES | |

| MOVLW | .0 | |
|---|---|---|
| MOVWF | ERR | ;SET ERR=0 TO START |

| | | |
|---|---|---|
| MOVWF | TXREG | ;MOVE FIRST ALL LOW BYTE INTO TRANSMIT REGISTER |

;
;ENABLE SERIAL PORT INTERRUPTS
;

| | |
|---|---|
| MOVLW | B‘00010000’ |
| MOVWF | PIE1 |

;
;

| | |
|---|---|
| MOVLW | .1 |

| | | | |
|---|---|---|---|
| LOOP1 | CPFSLT | TPOS | ;SEE IF WE HAVE HAD AN INTERRUPT, TPOS SHOULD BE |

= 0

| GOTO LOOP1 | | |
|---|---|---|
| MOVWF | TPOS | ;MAKE TPOS = 1 AGAIN FOR NEXT INTERRUPT |

;

| | | |
|---|---|---|
| MOVLW | .255 | ;NOW SEND ALL HIGH BYTE |
| MOVWF | TXREG | ;START SECOND BYTE |

;
;ENABLE SERIAL PORT INTERRUPTS
;

| | |
|---|---|
| MOVLW | B‘00010000’ |
| MOVWF | PIE1 |

;
;

| | | |
|---|---|---|
| MOVLW | .1 | ;MAKE READY TEST FOR INTERRUPT |

| | | | |
|---|---|---|---|
| LOOP2 | CPFSLT | TPOS | ;CHECK FOR TPOS = 0 AND SEE IF 2ND BYTE HAS LEFT |

BUFFER

| | GOTO LOOP2 | | |
|---|---|---|---|
| | MOVWF | TPOS | ;RESET TPOS TO 1 AGAIN |
| | MOVLW | 0X55 | ;SEND HEX 55 BIT RECOGNITION FOR AUTOBAUD |
| | MOVWF | TXREG | ;LOAD IT |
| ; | MOVLW | .1 | ;SET W REGISTER TO 1 AGAIN FOR TEST |
| ; | MOVWF | TPOS | ;GET READY FOR POSITION TEST FOR DATA |

;
;ENABLE SERIAL PORT INTERRUPTS
;

| | |
|---|---|
| MOVLW | B‘00010000’ |
| MOVWF | PIE1 |

;
;

| | | |
|---|---|---|
| MOVLW | .1 | ;SET W REGISTER TO 1 AGAIN FOR TEST |

| | | | |
|---|---|---|---|
| LOOP3 | CPFSLT | TPOS | ;INTERRUPT YET? |

| | |
|---|---|
| GOTO LOOP3 | ;NO LOOP |

;
;
;WE ARE DONE WITH INITIAL BYTES AND IT IS TIME FOR DATA
;
;DO TEMPERATURE FIRST

| | | |
|---|---|---|
| MOVWF | TPOS | ;MAKE TPOS = 1 AGAIN FOR NEXT INTERRUPT |

| | | |
|---|---|---|
| ; | goto LOOP | ;temporary loop |

| | | |
|---|---|---|
| MOVLW | B‘00001001’ | ;Channel 2 temperature first, Int voltage |

Ref, AD on

| MOVWF | ADCON0 | |
|---|---|---|
| BSF | ADCON0,GO | ;go bit starts conversion |

WAITT

| | | |
|---|---|---|
| BTFSC | ADCON0,GO | ;CONVERSION AND ACQUISTION DONE YET? |

| | |
|---|---|
| | ;REMEMBER SETTING ACQUISITION TIME MAKES THIS |
| EASY | |

| | |
|---|---|
| GOTO | WAITT |

;
;
;ENABLE SERIAL PORT INTERRUPTS
;

| | |
|---|---|
| MOVLW | B‘00010000’ |
| MOVWF | PIE1 |

;
;

| | |
|---|---|
| MOVLW | .1 |

| | | | |
|---|---|---|---|
| LOOP4 | CPFSLT | TPOS | ;SEE IF WE HAVE HAD AN INTERRUPT, TPOS SHOULD BE |

= 0

| GOTO LOOP4 | | |
|---|---|---|
| MOVWF | TPOS | ;MAKE TPOS = 1 AGAIN FOR NEXT INTERRUPT |

| | | |
|---|---|---|
| MOVFF | ADRESL,TXREG | ;SEND FIRST BYTE |

;
;ENABLE SERIAL PORT INTERRUPTS
;

| | |
|---|---|
| MOVLW | B‘00010000’ |
| MOVWF | PIE1 |

;
;

| | |
|---|---|
| MOVLW | .1 |

| | | | |
|---|---|---|---|
| LOOP5 | CPFSLT | TPOS | ;INTERRUPT YET? |

| GOTO LOOP5 | | ;WAIT UNTIL READY |
|---|---|---|
| MOVWF | TPOS | ;SET TPOS READY FOR NEXT INTERRUPT |

| | | |
|---|---|---|
| MOVFF | ADRESH,TXREG | ;START SECOND BYTE OF DATA |

;

| | | |
|---|---|---|
| DECF | TCOUN1 | ;TEMPERATURE HAS BEEN SENT, DECREASE COUNTER |

;SET UP NOW FOR ECG

| | | |
|---|---|---|
| DECF | TCOUN1 | ;DECREMENT AGAIN |

;

| | | |
|---|---|---|
| MOVLW | B‘00000001’ | ;CHANNEL 0, AD ON |
| MOVWF | ADCON0 | |

;
ECGLOOP
;

| | |
|---|---|
| BSF | ADCON0,GO ;START CONVERSION HERE |

WAIT2

| | |
|---|---|
| BTFSC | ADCON0,GO |
| GOTO | WAIT2 |

;
;
;ENABLE SERIAL PORT INTERRUPTS
;

| | |
|---|---|
| MOVLW | B‘00010000’ |
| MOVWF | PIE1 |

;
;
;
;GOT DATA NOW WAIT FOR INTERRUPT
;

| | |
|---|---|
| MOVLW | .1 |

| | | | |
|---|---|---|---|
| LOOP6 | CPFSLT | TPOS | ;INTERRUPT YET? |

| GOTO LOOP6 | | ;NO CONTINUE LOOPING |
|---|---|---|
| MOVWF | TPOS | ;RESET TPOS |

| | | |
|---|---|---|
| MOVFF | ADRESL,TXREG | ;YES SEND FIRST BYTE |

;
;
;ENABLE SERIAL PORT INTERRUPTS
;

| | |
|---|---|
| MOVLW | B‘00010000’ |
| MOVWF | PIE1 |

;
;

| | |
|---|---|
| MOVLW | .1 |

| | | | |
|---|---|---|---|
| LOOP7 | CPFSLT | TPOS | ;INTERRUPT YET |

| GOTO LOOP7 | | ;NO CONINUE LOOPING |
|---|---|---|
| MOVWF | TPOS | ;YES RESET AND CONTINUE |

;
;

| | | |
|---|---|---|
| MOVFF | ADRESH,TXREG | ;YES SEND SECOND BYTE |

;

| | |
|---|---|
| MOVLW | .0 |

| | | |
|---|---|---|
| CPFSGT | TCOUN1 | ;ALL DATA TAKEN FOR THIS PACKET? |
| GOTO LOOPEND | | |

| | |
|---|---|
| DECF | TCOUN1 |
| DECF | TCOUN1 |

| | |
|---|---|
| GOTO ECGLOOP | ;TAKE ANOTHER ECG POINT UNTIL 255 ARE TAKEN |

;

| | | |
|---|---|---|
| LOOPEND | GOTO LOOP | ;START WITH ANOTHER PACKET |

| | | | ;as it is set up there is no gap between data packets |
|---|---|---|---|
| ISR | | | ;entry point for interrupt routine |
| ; | | | |
| | MOVLW | 0x00 | ;TURN OFF INTERUPTS |
| | MOVWF | PIE1 | |

| | | |
|---|---|---|
| BTFSS | PIR1,TXIF | ;did transmit function cause interrupt? |

| | |
|---|---|
| GOTO otherint | ;no go to other interrupt service |

| | |
|---|---|
| MOVLW | .1 |

| | | |
|---|---|---|
| CPFSEQ | TPOS | ;Is this a valid interrupt? |

| | | |
|---|---|---|
| MOVWF | ERR | ;OH OH THIS MEANS AN INTERRUPT WITHOUT BEING READY; |
| CLRF | TPOS | ;YES, MAKE TPOS = 0 NOW AND GO TO RETURN |

GOTO ALLDONE

| | |
|---|---|
| otherint | ;NOTHING IMPLEMENTED AT THE MOMENT |

| | |
|---|---|
| MOVLW | .2 |
| MOVWF | ERR |

nop

ALLDONE

| | |
|---|---|
| | ;DO A RETURN FROM INTERRUPT WITH RESTORATION |
| (FAST) | |
| | ;OF REGISTERS BSR, STATUS AND WREG WITH NO |
| FURTHER CODE | |

| | |
|---|---|
| RETFIE FAST | ;FAST RETURN FROM INTERRUPT RESTORES WREG, BSR AND |

STATUS

NOP
END

1. An apparatus for monitoring a conscious subject in a magnetic field, the apparatus comprising:
a sensor adapted to detect a physiological parameter associated with the subject while being disposed in the magnetic field, the sensor being adapted to provide a sensed signal representative of the physiological parameter; and
a wireless transmitter responsive to the sensed signal, the wireless transmitter being adapted to wirelessly transmit a transmitted signal representative of the sensed signal while being disposed on the subject in the magnetic field, the apparatus being made substantially from non-ferromagnetic materials.

2. The apparatus defined by claim 1, wherein the sensor is adapted to detect at least one of an electrocardiogram (EKG) signal, electroencephalogram (EEG) signal, electromyogram (EMG) signal, electrooculogram (EOG) signal, pulse oximetry, respiration, blood pressure, and temperature.

3. The apparatus defined by claim 1, further comprising an amplifier responsive to the sensed signal.

4. The apparatus defined by claim 1, further comprising a filter responsive to the sensed signal.

5. The apparatus defined by claim 4, further comprising a filter responsive to the sensed signal, the filter being adapted to pass a frequency range of substantially 1.4 Hz to 30.0 Hz.

6. The apparatus defined by claim 1, further comprising a processing device responsive to the sensed signal, the processing device comprising at least one of a microcontroller, microprocessor, application specific integrated circuit (ASIC), field programmable gate array (FPGA), and programmable device.

7. The apparatus defined by claim 1, further comprising a processing device responsive to the sensed signal, the processing device being adapted to perform at least one of digitizing information associated with the sensed signal, packetizing information associated with the sensed signal, and formatting information associated with the sensed signal.

8. The apparatus defined by claim 1, further comprising an analog-to-digital converter responsive to the sensed signal.

9. The apparatus defined by claim 1, further comprising a wireless receiver adapted to wirelessly receive a received signal while being disposed on the subject in the magnetic field.

10. The apparatus defined by claim 1, wherein the subject comprises one of an animal and a human.

11. The apparatus defined by claim 1, wherein the subject comprises a rat.

12. The apparatus defined by claim 1, wherein the transmitted signal comprises a frequency of substantially 916.5 Mhz.

13. The apparatus defined by claim 9, wherein the received signal comprises a frequency of substantially 916.5 Mhz.

14. The apparatus defined by claim 1, wherein the magnetic field is generated by a magnetic resonance imaging (MRI) scanner.

15. The apparatus defined by claim 1, wherein the magnetic field comprises a field strength of at least 0.5 Tesla.

16. The apparatus defined by claim 1, wherein the apparatus is made substantially from at least one of non-metallic, non-ferrous, non-ferritic, and non-magnetic materials.

17. A system for monitoring a conscious subject in a magnetic field, the apparatus comprising:
a wireless monitor comprising:
a sensor adapted to detect a physiological parameter associated with the subject while being disposed in the magnetic field, the sensor being adapted to provide a sensed signal representative of the physiological parameter, the sensor being made substantially from non-ferromagnetic materials; and
a wireless transmitter responsive to the sensed signal, the wireless transmitter being adapted to wirelessly transmit a first transmitted signal representative of the sensed signal while being disposed on the subject in the magnetic field, the wireless transmitter being made substantially from non-ferromagnetic materials;
a wireless interface adapted to wirelessly receive the first transmitted signal and transmit a second transmitted signal representative of the first transmitted signal; and
a computer adapted to receive the second transmitted signal.

18. The system defined by claim 17, wherein the computer is adapted to at least one of process, tabulate, graph, display, analyze, detect errors, correct errors, filter, and format information associated with the second transmitted signal.

19. The system defined by claim 17, wherein the wireless interface is adapted to transmit a third transmitted signal, the wireless monitor further comprising a wireless receiver adapted to wirelessly receive the third transmitted signal from the wireless interface while being disposed on the subject in the magnetic field, the wireless receiver being made substantially from non-ferromagnetic materials.

20. The system defined by claim 17, wherein at least one of the first and second transmitted signals comprises a frequency of substantially 916.5 Mhz.

21. The system defined by claim 17, wherein the third transmitted signal comprises a frequency of substantially 916.5 Mhz.

22. The system defined by claim 17, wherein the wireless monitor is made substantially from at least one of non-metallic, non-ferrous, non-ferritic, and non-magnetic materials.

23. A method of monitoring a conscious subject in a magnetic field, the method comprising:
disposing a sensor in the magnetic field, the sensor being made substantially from non-ferromagnetic materials;
sensing a physiological parameter associated with the subject by the sensor;
providing a sensed signal representative of the physiological parameter from the sensor;
disposing a wireless transmitter responsive to the sensed signal on the subject in the magnetic field, the wireless transmitter being made substantially from non-ferromagnetic materials; and
transmitting a first transmitted signal representative of the sensed signal from the wireless transmitter.

24. The method defined by claim 23, wherein sensing the physiological parameter further comprises sensing at least one of an electrocardiogram (EKG) signal, electroencephalogram (EEG) signal, electromyogram (EMG) signal, electrooculogram (EOG) signal, pulse oximetry, respiration, blood pressure, and temperature.

25. The method defined by claim 23, further comprising amplifying the sensed signal.

26. The method defined by claim 23, further comprising filtering the sensed signal.

27. The method defined by claim 23, further comprising filtering the sensed signal to pass a frequency range of substantially 1.4 Hz to 30.0 Hz.

28. The method defined by claim 23, further comprising processing the sensed signal, the processing comprising at least one of digitizing information associated with the sensed signal, packetizing information associated with the sensed signal, and formatting information associated with the sensed signal.

29. The method defined by claim 23, further comprising generating the magnetic field by a magnetic resonance imaging (MRI) scanner.

30. The method defined by claim 23, further comprising:
receiving the first transmitted signal by a wireless interface;
transmitting a second transmitted signal representative of the first transmitted signal from the wireless interface; and
receiving the second transmitted signal by a computer.

31. The method defined by claim 30, further comprising adapting the computer to perform at least one of processing, tabulating, graphing, displaying, analyzing, detecting errors, correcting errors, filtering, and formatting information associated with the second transmitted signal.

32. The method defined by claim 30, further comprising:
transmitting a third transmitted signal by the wireless interface;
disposing a wireless receiver on the subject in the magnetic field, the wireless receiver being made substantially from non-ferromagnetic materials; and
receiving the third transmitted signal by the wireless receiver.

33. The method defined by claim 23, further comprising making at least one of the sensor and wireless transmitter substantially from at least one of non-metallic, non-ferrous, non-ferritic, and non-magnetic materials.

34. The method defined by claim 32, further comprising making the wireless receiver substantially from at least one of non-metallic, non-ferrous, non-ferritic, and non-magnetic materials.
